(19) United States
(12) Patent Application Publication
(10) Pub. No.: US 2025/0186310 A1
(43) Pub. Date: Jun. 12, 2025

(54) SUN PROTECTION FACTOR (SPF) MINERAL POWDER BLOTTING PAPER

(71) Applicant: Connie Michele Nerio, New York, NY (US)

(72) Inventor: Connie Michele Nerio, New York, NY (US)

(21) Appl. No.: 18/963,519

(22) Filed: Nov. 28, 2024

Related U.S. Application Data
(60) Provisional application No. 63/607,581, filed on Dec. 8, 2023.

(51) Int. Cl. A61K 8/02; A61Q 17/04

(52) U.S. Cl. CPC A61K 8/0208; A61Q 17/04; A61K 2800/262; A61K 2800/87

(57) ABSTRACT

The present invention relates to a method for applying sun protection to a user's skin. The method involves disposing a sun protection factor (SPF) layer on a first surface of a paper. The paper is then retained in a refillable container and dispensed from an opening of the container. To apply the sun protection, the SPF layer of the paper is pressed against the user's skin, thereby transferring at least a portion of the SPF layer onto the user's skin. This method provides a convenient and portable way to apply sun protection, allowing users to easily access and apply SPF whenever needed.

### CROSS-REFERENCE TO RELATED APPLICATIONS

The present application claims priority under 35 USC 119 (e) from U.S. Provisional Application No. 63/607,581 filed on Dec. 8, 2023, the entire contents of which are incorporated by reference.

### BACKGROUND

The present disclosure relates generally to a combination product that incorporates sun protection with oil-absorbing properties. Various approaches have been developed for providing sun protection to individuals, including the use of lotions, creams, sprays, and other topical formulations. These conventional methods typically involve applying a sun protection factor (SPF) product directly onto the skin to provide a barrier against harmful ultraviolet (UV) radiation from the sun. However, these conventional methods often require the user to carry a separate container of the SPF product, which can be inconvenient and may result in the product being forgotten or misplaced.

In an effort to address these challenges, some approaches have involved incorporating SPF into various substrates, such as fabrics, films, and papers. For example, certain SPF-infused fabrics have been developed to provide sun protection while also offering comfort and breathability. Similarly, SPF films have been used in the production of stickers or patches that can be adhered to the skin for sun protection. However, these approaches have limitations, as they often require separate packaging or application methods, which can be cumbersome and may result in the SPF product being wasted or improperly applied.

Additionally, there have been attempts to develop refillable containers for SPF products to enhance convenience and reduce waste. These containers typically include a reservoir for holding the SPF product and a dispensing mechanism for controlled release. However, these refillable containers have primarily been designed for use with conventional SPF formulations and have not addressed the challenges associated with incorporating SPF into a substrate for direct application to the skin.

In summary, while various approaches have been proposed for providing sun protection, none of these approaches have provided a comprehensive solution that combines the features described in this disclosure. There remains a need for a method that allows for convenient and efficient application of SPF to the skin using a refillable container and a substrate that incorporates the SPF, thereby eliminating the need for separate packaging and improving user experience.

### SUMMARY OF THE INVENTION

In some aspects, the techniques described herein relate to a method including: disposing a sun protection factor (SPF) layer at a first surface of a paper; retaining the paper in a refillable container; dispensing the paper from an opening of the refillable container; and pressing the SPF layer of the paper against a user's skin to apply at least a portion of the SPF layer to the user's skin.

In some aspects, the techniques described herein relate to a method wherein the SPF layer is a mineral powder. 3 The method wherein the SPF layer is a translucent film.

In some aspects, the techniques described herein relate to a sun protection factor (SPF) blotting paper including: a paper material; and a SPF layer is disposed at a surface of the blotting paper.

In some aspects, the techniques described herein relate to a SPF blotting paper wherein the SPF layer is a mineral powder.

In some aspects, the techniques described herein relate to a SPF blotting paper wherein the SPF layer is a translucent film.

### BRIEF DESCRIPTION OF THE DRAWINGS

The present disclosure may be better understood, and its numerous features and advantages made apparent to those skilled in the art by referencing the accompanying drawings. The use of the same reference symbols in different drawings indicates similar or identical items.

FIG. 1 is a flowchart illustrating a method, according to some embodiments of the present disclosure.

FIG. 2 is a block diagram illustrating a sun protection factor, according to some embodiments of the present disclosure.

### DETAILED DESCRIPTION

SPF mineral powder blotting paper is a multifunctional cosmetic product that combines sun protection, oil absorption, and skin mattification in a convenient form. The SPF mineral powder component comprises finely milled mineral-based particles like zinc oxide or titanium dioxide. These minerals possess the ability to block or scatter UV rays when applied to the skin, providing sun protection. The powder format ensures ease of application and reapplication throughout the day without the heavy or greasy feel often associated with traditional sunscreen lotions. The blotting paper component consists of an absorbent material designed to absorb excess oil, sebum, or shine from the skin's surface. Blotting papers are effective at removing surface oils without disturbing makeup, leaving the skin with a matte appearance. Combining SPF mineral powder with blotting paper offers a dual-action benefit. When used, the mineral powder dusted onto the skin provides a protective barrier against harmful UV rays. Simultaneously, the blotting paper component helps manage and reduce surface oil, offering a matte finish and preventing excessive shine on the skin.

The product's design caters to individuals seeking convenient sun protection without the drawbacks of conventional liquid or cream sunscreens. However, the efficacy of SPF mineral powder blotting paper in providing sun protection may vary based on factors like application technique, coverage, and the frequency of reapplication. Therefore, while it offers a practical solution, comprehensive sun protection should involve combining this product with other measures such as broad-spectrum sunscreen application, protective clothing, and minimizing direct sun exposure during peak hours.

An integration of blotting paper and mineral powder has not been previously explored for sun protection. Typically, blotting paper primarily functions to absorb excess oils without incorporating sun protection properties. Concurrently, there exists an absence of utilizing blotting paper as a vehicle for the application of sun protection. Conversely, mineral powders possess inherent oil-absorbing qualities while concurrently acting as a natural mineral-based sunscreen by forming a physical barrier against ultraviolet (UV) rays. Despite these individual functionalities, the synergistic amalgamation of blotting paper's oil-absorbing capacity with the UV protection capabilities inherent in mineral powders remains unexplored.

The SPF mineral powder is formulated to be a translucent product and involves a unique contrast between a paper substrate coated with a dry mineral sunscreen formulation compared to a towelette infused with liquid sunscreen. Notably, unlike the liquid-infused towelette, the mineral-coated paper does not necessitate a protective barrier packaging film to maintain moisture or prevent desiccation. While both portable, the distinctiveness of this offering lies in the application method: pressing paper coated with mineral powder onto the skin to create a protective, translucent SPF film, in contrast to the liquid product applied by wiping. This methodology allows the product's use over makeup due to its dry nature and delivery mechanism, wherein the pressing action deposits a translucent SPF film atop existing makeup. In contrast, a liquid product applied with a towelette might cause makeup smearing. Dispensing the sunscreen-coated paper occurs through a proprietary refillable container design, featuring a patent-pending mechanism. This design not only safeguards the blotting paper but also minimizes waste, enhancing its eco-friendliness through reduced wastage.

The application involves pressing mineral powder-coated paper against the skin, creating a protective SPF translucent film, eliminating the necessity for a brush or for a sponge or the manual application method required by liquid sunscreen. Unlike this product or comparable items, my product enables users to apply it over makeup, allowing for SPF refreshment throughout the day. Dispensing the sunscreen-coated paper utilizes a refillable container, showcasing an eco-friendly approach. Conversely, single-use plastic packets lack eco-friendliness. Additionally, the product, being a coated paper, mitigates the risk of liquid spillage, unlike single-use plastic packets which can be punctured and lack resealable properties to prevent leaks if the contents are not entirely used. Dispensing the sunscreen-coated paper occurs through a compact refillable container, offering a more compact and streamlined alternative compared to carrying a bulky powder tube with a brush, allowing effortless portability in pockets or small bags.

The coated paper product boasts a non-sticky texture and exhibits reduced susceptibility to contamination. Conversely, items featuring a “sticky” texture, such as sunscreen sticks, are prone to contamination when lids are inadequately sealed. This exposure can result in the product surface being susceptible to debris like crumbs or lint in pockets or bags. Opting for the coated paper as a single-use solution contrasts with reusing solid products, which can face contamination from skin or finger-borne bacteria or exposure to lint if their lids loosen within pockets or bags.

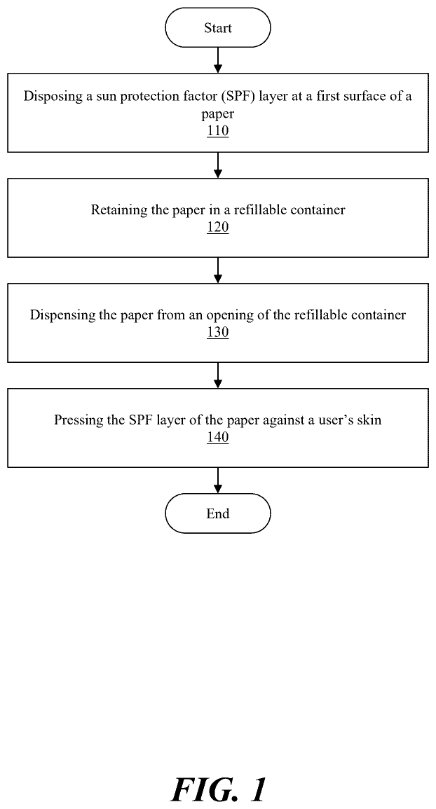

FIG. 1 is a flowchart that describes a method, according to some embodiments of the present disclosure. In some embodiments, at 110, the method may include disposing a sun protection factor (SPF) layer at a first surface of a paper. At 120, the method may include retaining the paper in a refillable container. At 130, the method may include dispensing the paper from an opening of the refillable container. At 140, the method may include pressing the SPF layer of the paper against a user's skin to apply at least a portion of the SPF layer to the user's skin. In some embodiments, the SPF layer may be a mineral powder. In some embodiments, the SPF layer may be a translucent film. In some embodiments, the SPF layer may be a mineral powder. In some embodiments, the SPF layer may be a translucent film.

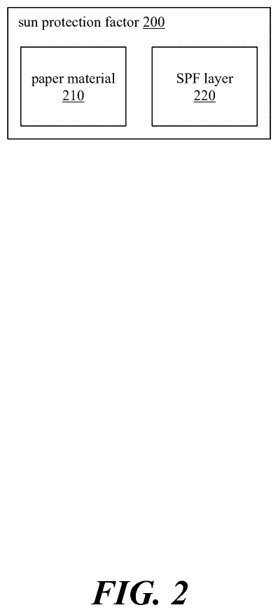

FIG. 2 is a block diagram that describes a sun protection factor 200, according to some embodiments of the present disclosure. In some embodiments, the sun protection factor 200 may include a paper material 210 and a SPF layer 220 may be disposed at a surface of the blotting paper.

Note that not all of the activities or elements described above in the general description are required, that a portion of a specific activity or device may not be required, and that one or more further activities may be performed, or elements included, in addition to those described. Still further, the order in which activities are listed are not necessarily the order in which they are performed. Also, the concepts have been described with reference to specific embodiments. However, one of ordinary skill in the art appreciates that various modifications and changes can be made without departing from the scope of the present disclosure as set forth in the claims below. Accordingly, the specification and figures are to be regarded in an illustrative rather than a restrictive sense, and all such modifications are intended to be included within the scope of the present disclosure.

Benefits, other advantages, and solutions to problems have been described above with regard to specific embodiments. However, the benefits, advantages, solutions to problems, and any feature(s) that may cause any benefit, advantage, or solution to occur or become more pronounced are not to be construed as a critical, required, or essential feature of any or all the claims. Moreover, the particular embodiments disclosed above are illustrative only, as the disclosed subject matter may be modified and practiced in different but equivalent manners apparent to those skilled in the art having the benefit of the teachings herein. No limitations are intended to the details of construction or design herein shown, other than as described in the claims below. It is therefore evident that the particular embodiments disclosed above may be altered or modified and all such variations are considered within the scope of the disclosed subject matter. Accordingly, the protection sought herein is as set forth in the claims below.

What is claimed is:

1. A method comprising:
disposing a sun protection factor (SPF) layer at a first surface of a paper;
retaining the paper in a refillable container;
dispensing the paper from an opening of the refillable container; and
pressing the SPF layer of the paper against a user's skin to apply at least a portion of the SPF layer to the user's skin.

2. The method of claim 1 wherein the SPF layer is a mineral powder.

3. The method of claim 1 wherein the SPF layer is a translucent film.

4. A sun protection factor (SPF) blotting paper comprising:
a paper material; and
a SPF layer is disposed at a surface of the blotting paper.

5. The SPF blotting paper of claim 1 wherein the SPF layer is a mineral powder.

6. The SPF blotting paper of claim 1 wherein the SPF layer is a translucent film.
